(19) United States
(12) Patent Application Publication
(10) Pub. No.: US 2022/0040447 A1
(43) Pub. Date: Feb. 10, 2022

(54) Multi-Lumen Intravascular Catheters with inner Converging Lumens for Multiple Guidewire Control

(71) Applicant: Mekal LLC, Milwaukee, WI (US)

(72) Inventor: Mark W. Mewissen, Franklin, WI (US)

(73) Assignee: Mekal LLC, Milwaukee, WI (US)

(21) Appl. No.: 17/369,008

(22) Filed: Jul. 7, 2021

Related U.S. Application Data
(60) Provisional application No. 63/061,218, filed on Aug. 5, 2020.

(51) Int. Cl. A61M 25/00; A61M 25/09

(52) U.S. Cl. CPC A61M 25/0028; A61M 25/09; A61M 2025/0177; A61M 2205/02; A61M 2025/0098; A61M 25/003

(57) ABSTRACT

Catheter systems with multiple inner lumens that converge at the catheter distal tip are preloaded with at least two guidewires. The catheters offers sequential probing with two or more guidewires, therefore improving on procedural speed and accuracy. The catheters comprise proximal and distal regions enabling angled lumens to merge into a common lumen with a diameter slightly larger to the largest of the two proximal lumens. The confluence of the two proximal lumens comprises a single lumen with a reduced profile and cross sectional area. The lumens merge with the larger lumen at an angle greater than zero degrees, and the distal tip has a diameter equal to the diameter of the smaller proximal lumen. The proximal region serves as a directed passageway for different guidewires that enter the catheter through two proximal lumens and which may be reoriented through a convergence region for sequential exiting of the catheter tip.

### CROSS-REFERENCE TO RELATED APPLICATION

This utility application is based upon, and claims the benefit of the filing date of prior pending U.S. Provisional application, Ser. No. 63/061,218, entitled “Catheter system with multiple inner lumens that converge into one lumen at the distal tip of the catheter,” filed Aug. 5, 2020, by inventor Mark William Mewissen (American Citizen).

### BACKGROUND OF THE INVENTION

### 1. Field of the Invention

The present invention relates generally to apparatus and intravascular methods for inserting vascular catheters into blood vessels, including multi-lumen catheters which are suitable for procedures using multiple guidewires of different diameters. More specifically, the present invention relates to multiple lumen catheters which allow rapid tracking and steering of the catheter and the guidewires therein to a vascular target while facilitating and enhancing the direction of catheter portions into particular blood vessels or surgical targets.

### 2. Description of the Prior Art

A wide variety of percutaneous endovascular procedures exist in which a catheter or other miniature instrument is inserted percutaneously into a blood vessel to treat a vascular disease or condition. Examples of commonly performed endovascular procedures to treat peripheral vascular disease (PVD) may include the insertion of a catheter into a narrowed or occluded vessel so as to open the blockage with a balloon, a metallic stent, an atherectomy catheter, a laser catheter or any other device designed to open the blockage. All vascular procedures begin with a vascular guidewire. Guidewires are the lead device when treating any narrowing or total blockage in the vascular system. In general, most endovascular treating devices require safe passage of a guidewire across the lesion to provide support and over the wire tracking of the device through the target lesion. This fundamental step of traversing a lumen target with a guidewire can be challenging particularly when treating chronic total occlusions (difficult vascular blockages).

Guidewires are produced in three common diameters, 0.014, 0.018 and 0.035 inches, and they may be classified by the weight of the distal tip portion, with the weight expressed in grams. There are presently in excess of one hundred guidewires available in today's market, all with different specifications and characteristics. As a guidewire exits the distal tip of a support catheter to probe a difficult vascular blockage, it is important that the guidewire retain all of its characteristics. For instance, torque control and shape can be lost in tortuous applications, thereby denigrating the ability to cross difficult lesions. As guidewire technology becomes more evolved, operators continue to pursue the endovascular treatment of increasingly challenging vascular blockages. Guidewire support catheters have evolved in this arena as a compliment to guidewires in assisting the operator to cross increasingly difficult anatomy.

There are a great number of single lumen and multiple lumen guidewire support catheters currently available. No known prior art catheter is specifically designed to fully retain the tracking guidewire characteristics, such as guidewire weight and tip shape to control and change the direction of the distal tip. guidewire to help support the catheter and to negotiate difficult anatomy. The current art only offers support (i.e., stiffness) to the guidewire.

Typically, a single lumen diagnostic catheter is tracked to the target lesion, through a vascular sheath added for support. Once the catheter reaches a difficult vascular blockage, a guidewire of choice is passed through the catheter lumen and extended from its distal tip until it reaches the target to be treated. Probing of the lesion is initiated with the guidewire to penetrate the blockage while remaining in the true lumen. The operator rotates the guidewire to transfer torque while advancing the diagnostic catheter into the blockage. Because difficult vascular blockages are usually long, severely calcified lesions with a proximal fibrous cap, failure to pass the initial guidewire completely across the lesion is very common.

Furthermore, the guidewire can take an unwanted track into a subintimal space outside the true lumen or dissection plane, making it more difficult to get back to the proper track and reach the true lumen beyond the blockage. As a result, several guidewire exchanges may be necessary to successfully penetrate the entire length of the lesion, with each guidewire serving different functions. However, when the second guidewire is exchanged for and inserted within the blocked lumen, it is likely that it will follow the same path as the first guidewire. Because single lumen catheters accommodate only one guidewire at a time, the multiple guidewire exchanges are lengthy and labor intensive. Because the distal tip of existing single lumen catheters used to cross difficult vascular blockages is not usually designed to enhance its crossing profile or its steerability, repetitive passage of the different guidewires into the same path or subintimal space is common, with resultant unsuccessful re-entry into the reconstituted lumen distal to the vascular blockage. This strategy of wire escalation with a single lumen support catheter is very common, and when unsuccessful, it can lead to longer procedures, increased radiation exposure, and increased complications such as the need for amputation.

Thus, a need exists for a dexterous catheter capable of use with multiple guidewires employed in treating various intravascular phenomena. In particular, a catheter configuration is needed that alleviates problems occasioned when a first guidewire has been directed outside of the vessel lumen, or which cannot be advanced beyond the blockage. An appropriate catheter should increase the likelihood of a successful crossing of intravascular blockages. Moreover, any proposed solution should be adaptable for use with a variety of catheter types and configurations.

Thus, it would be highly advantageous to have a dual guidewire catheter system that avoids the above limitations and solves the above-discussed limitations. No prior art known at this time exists for a multi-lumen catheter that accommodates and supports guidewires of various diameters.

Within the prior art numerous examples of multiple lumen catheters, and guidewire-enabling catheters exist. For example, U.S. Pat. No. 3,459,184 issued Aug. 5, 1969 to W. Ring shows a needle guiding catheter lumen.

U.S. Pat. No. 4,306,562 issued Dec. 22, 1981 discloses a tear apart apart cannula with twin lumens. Body portions tear in a longitudinal direction for easy removal removal by pulling tabs on opposite sides of the cannula after the catheter has been inserted into the body.

U.S. Pat. No. 4,484,585 issued Nov. 27, 1984 discloses a catheter for measuring the pressure along the length of a patient's urethra. A lateral outlet spaced apart from a closed distal end admits fluid. The catheter is transversely divided into two parts constituting distal and proximal sections having mutually opposed terminal areas. These terminal areas are connected by a rigid bridging member to form an annular gap, which latter for example has a width of advantageously between 0.2 and 0.5 mm.

U.S. Pat. No. 4,493,696 issued Jan. 15, 1985 provides a double lumen cannula for hemodialysis and subclavian insertion. Elongated flexible tubular members, disposed one within the other, are adapted to be inserted within the subclavian vein of a patient, leaving semi-permanent access thereto for repeated dialysis treatments. The cannula is formed of an elongated flexible outer member, having a smooth exterior surface, and made up of an elongated main portion, a convergent, constricting portion and a narrower distal end portion which is tapered at the tip to fit over a Seldinger guidewire.

U.S. Pat. No. 4,641,912 issued Feb. 10, 1987 discloses an Excimer laser delivery system, for angioscope and angioplasty surgery. In such an application, the delivery system can be used to transmit both laser and illuminating light so as to reduce the number of optical fibers that are required and thereby provide a system that is sufficiently small to be fed into a coronary artery. An ultra-thin image scope facilitates a determination of the distance of the distal end from the viewed object and hence the size of the object.

U.S. Pat. No. 4,769,005 issued Sep. 6, 1988 shows guiding sheath for inserting guidewires or vascular catheters within a blood vessel includes an elongate body having plurality of axial lumens. At least some of the lumens project laterally from the body at preselected angles so that, when inserted therethrough, the guidewire or catheter will exit at said preselected angle to facilitate directing the guidewire or catheter to the proper location within the vascular system. Usually, the insertional sheath will include a primary axial lumen which is used for inserting the sheath on a guidewire to a location where a branching in the vascular system occurs. The sheath is then positioned under fluoroscopic guidance so that one of the lumens is then properly located to direct the guidewire or catheter in the desired direction.

U.S. Pat. No. 4,968,307 issued Nov. 6, 1990 discloses another vascular catheter for delivering therapeutic and/or thrombolytic agents to a thrombus or clot in a patient's arterial system. The catheter has a relatively thick-walled tubular body with a central lumen for advancement over a guidewire and a plurality of fluid delivering lumens. Each of the smaller lumens has a single flow passageway to discharge therapeutic fluid to the exterior of the catheter.

U.S. Pat. No. 5,195,962 issued Mar. 23, 1993 discloses a triple lumen catheter with a flexible body having a distal end with a tapered tip, a proximal end, an outer wall and a septum extending between spaced points on the outer wall. The outer wall and the septum define extraction and return lumens extending from the proximal end to the tapered tip where the outer wall and the septum converge to close off the lumens. A portion of the septum defines a third lumen extending along the longitudinal axis of the body from the proximal end to the distal end and terminating at the tip in an aperture. This third lumen is useful to receive a Seldinger wire for insertion and can be used also for intravenous infusion of liquid medicaments.

U.S. Pat. No. 5,320,605 issued Jun. 14, 1994 discloses a multi-wire multi-balloon catheter for administering treatments to stenotic regions. A two wire catheter system is disclosed, along with a multiple wire, multiple balloon system. Additional balloons and associated catheters may also be incorporated into the multiple wire multiple balloon system. Methods are also discussed for accomplishing sequential dilatations using a multiple wire multiple balloon catheter.

U.S. Pat. No. 5,397,302 issued Mar. 14, 1995 discloses a biliary catheter comprising a polyurethane or nylon tube with a hydrophilic coating providing lubricity, kink resistance and suppleness. The tube contains a first crescent-shaped lumen channel extending between the proximal and the distal end for transporting contrast medium from to the biliary duct; and a second circular lumen channel extending between the proximal end and distal end facilitating the insertion and threading of a spring wire guide.

U.S. Pat. No. 6,494,846 issued Dec. 17, 2002 discloses a dual mode catheter allowing manipulation of stents, balloons, other medical devices, or combinations thereof. Thus combined, the present invention allows greater control of the medical devices and the catheter by a single physician while simultaneously allowing distal injection of individual components such as iodinated drugs.

U.S. Pat. No. 6,689,157 issued Feb. 10, 2004 discloses is a dual lumen catheter facilitating placement of two procedure wires across a treatment site. In one application, the catheter is used to place a first wire extending between a contralateral iliac and an ipsilateral iliac across the terminal bifurcation of the aorta, and a second wire extending through a portion of the ipsilateral iliac and into the aorta.

U.S. Pat. No. 6,746,466 issued Jun. 8, 2004 discloses a wire station for securing and managing multiple wires used in a catheter. The station secures the proximal portion of two wire members in a spatially separate arrangement. The wire station additionally allows either the direct attachment of the wire station to the catheter, or the positioning of the wire station at a remote location.

U.S. Pat. No. 6,849,077 issued Feb. 1, 2005 discloses a balloon dilation catheter comprising a tubular member having a proximal end and a distal end that supports an inflatable balloon tubular member. A first lumen is communication with the balloon interior. A second lumen is disposed in the tubular member for receiving a guidewire. The second lumen has a first opening in the proximal region of the tubular member and a second opening at the distal region of the tubular member. The balloon dilation catheter provides improved exchange advantages of either the catheter or the guidewire used in a catheterization technique.

U.S. Pat. No. 7,001,358 issued Feb. 21, 2006 shows a reinforced monorail balloon catheter comprising an elongated shaft, a guidewire tube, and an angioplasty balloon. The distal end of the balloon is attached to the distal portion of the guidewire tube, and the proximal portion of the balloon is attached to the distal portion of the catheter shaft. A stiffening wire is attached to the inner wall of the catheter shaft at a plurality of points along its length. The distal extremity of the guidewire tube is decreased in size relative to the proximal portion of the guidewire tube. A fluid is contained within the guidewire lumen.

U.S. Pat. No. 7,204,831 issued Apr. 17, 2007 reveals an apparatus for passing a guidewire through a subcutaneous tissue tunnel to enable over-the wire insertion of a catheter through the tissue tunnel. An elongated member having a distal end portion comprises a tip configured to advance through the tissue tunnel and an opening extending longitudinally with respect to the apparatus and dimensioned to receive the guidewire. The guidewire is inserted through the opening to pass the guidewire through the tunnel to enable subsequent insertion of the catheter.

U.S. Pat. No. 7,846,127 issued Dec. 7, 2010 discloses a multi lumen catheter including an inner tube having a front tip, an outer tube, a tube or lumen for inserting a guidewire, a blood extraction lumen and a blood return lumen which are formed in the outer tube and inner tube, respectively, wherein the inner tube slides relative to the outer tube. A multi-lumen catheter includes a balloon provided on a tabular body, a blood return port and a blood extraction port provided in the tubular body on opposite sides of the balloon, and an outer cylinder slidable on the tubular body to close the blood extraction and blood return ports and the balloon.

U.S. Pat. No. 8,104,483 issued Jan. 31, 2012 discloses a multi-port light delivery catheter allowing the advance of a guidewire through a first proximal lumen and a distal lumen of the catheter into a first region of an occlusion, retraction of the guidewire from the distal lumen, and advancement of a diagnostic device from a second proximal lumen of the catheter to examine the occlusion.

U.S. Pat. No. 8,206,370 issued Jun. 26, 2012 discloses a dual lumen guidewire support catheter for crossing a chronic total occlusion in a vessel. A first lumen and a second lumen share a common side and a common distal end. The first lumen allows rapid exchange, while the second lumen is an over the wire portion. Both lumens include a discontinuity enabling removal of a guidewire following placement through or at an occlusion or lesion.

U.S. Pat. No. 8,617,231 issued Dec. 31, 2013 discloses a dual guidewire exchange catheter system comprising two lumens for receiving main and branch guidewires. The catheter includes an exit port for each guidewire, at least one of which is a shorter distance from the distal end of the catheter system than the distance from the distal end to the proximal end of the catheter. This allows for management of relatively short guidewire lengths outside the body.

U.S. Pat. No. 8,628,519 issued Jan. 14, 2014 discloses balloon biasing lasers catheters that may include a distal tip that extends from the distal end of the catheter from a point near light guide aperture. The distal tip may be disposed at the periphery of the catheter. In some embodiments, a balloon may be disposed between the light guide aperture and the distal tip, such that the a light guide extending from the aperture may be disposed proximate with the distal tip having the balloon in between. A retaining wire may keep the light guide biased relatively parallel with the distal tip and/or the catheter body when the balloon is inflated. The light guide may include a guidewire lumen the extends to the distal end of the distal tip.

U.S. Pat. No. 9,931,166 issued Apr. 3, 2018 discloses offset catheters that may include a guidewire tube and an elastic rib that provides an offset or separation between the catheter and the guidewire tube in its resting state. The rib has an initial resting state, but may be forced into a compressed state. When released from the compressed state, the rib returns to its resting state. An offset catheter may be compressed and slid through a sheath. When the offset catheter emerges from the sheath, the distal tip will return to its resting state providing an operation offset.

U.S. Pat. No. 10,130,385 issued Nov. 20, 2018 discloses a catheter for insertion into vasculature of a patient to a target area includes a hollow inner shaft, a non-occluding self-expandable scaffold coupled to the distal end of the inner shaft and disposed at the distal end of the inner shaft, and a hollow outer shaft. The outer shaft is slidable over the inner shaft and scaffold such that the scaffold is in a non-expanded state when the outer shaft is around the scaffold.

U.S. Pat. No. 10,130,795 issued Nov. 20, 2018 shows a catheter with telescoping lumens for passing one or more guidewires (via the use of one or more telescoping guidewire lumens) through a chronic total occlusion of a vasculature. The catheter may include a shaft having a distal end, one or more telescoping guidewire lumen catheters passing longitudinally through the shaft, an expansible distal portion, and a retractable sheath that can activate the expansible distal portion of the shaft causing expansion.

U.S. Pat. No. 10,426,510 issued Oct. 1, 2019 reveals methods and apparatus for centering a micro-catheter within a vasculature for enabling a guidewire to cross through a chronic total occlusion (i.e., “CTO”).

U.S. Pat. No. 10,471,234 issued Nov. 12, 2019 reveals a catheter with a flexible shaft, guidewire lumens passing through the shaft, and a positioning device for positioning the guidewires relative to an external lumen. The positioning device may be an expansible scaffold covered with a retractable sheath. The positioning device may also be one or more balloons that are inflated through an inflation port in the shaft.

U.S. Pat. No. 10,518,064 issued Dec. 31, 2019 discloses another catheter with a multi-lumen configuration for use in accessing a vasculature of a patient. The catheter assembly includes a catheter body with a flattened, oval outer surface, and first and second lumens. The catheter body defines a distal tip region that includes a venous lateral opening that is in fluid communication with the first lumen and includes a distal-facing portion. The distal tip region further includes an arterial lateral opening that is in fluid communication with the second lumen, includes a distal-facing portion, and is substantially un-staggered with respect to the venous lateral opening.

U.S. Pat. No. 10,716,690 issued Jul. 21, 2020 discloses a system for deploying a prosthesis between an ipsilateral lumen and a contralateral lumen that includes a guidewire, a guidewire capture catheter, a self-expanding tubular prosthesis, and a delivery catheter. The guidewire capture catheter pulls the guidewire out through the contralateral side, and the guidewire is used to advance a delivery catheter from the ipsilateral side.

U.S. Pub. No. 20020165571 published Nov. 7, 2002 discloses a a delivery catheter with a flexible, proximally-manipulated hinge or joint region. The inventive catheter and an optional balloon region. An inner delivery, lumen may be used with a guidewire to access target sites within the body via the flexible, small diameter vessels of the body. The delivery lumen may be also used for placement of occlusive materials, e.g., in an aneurysm. Inflation of a micro-balloon, located near the distal tip of the catheter, is effected using the inflation lumen. The delivery catheter may be capable of twisting in a helical or corkscrew-like manner for traversing certain vasculature.

U.S. Pub. No. 20040098087 published May 20, 2004 discloses a dual lumen access catheter allowing placement of two procedure wires across a treatment site. In one application, the catheter is used to place a first wire extending between a contralateral iliac and an ipsilateral iliac across the terminal bifurcation of the aorta, and a second wire extending through a portion of the ipsilateral iliac and into the aorta.

U.S. Pub. No. 20080009770 published Jan. 10, 2008 discloses a stent delivery and guidance system for controlling multiple guidewires. It comprises a guide rail, which has a housing that defines a first guidewire lumen and a second guidewire lumen.

U.S. Publication No. 20080009803 published Jan. 10, 2008 discloses a multi-lumen catheter including a unitary portion and at least two distal end tubes extending away from the unitary portion. The unitary portion includes at least two distal end tubes.

U.S. Pub. No. 20090326450 published Dec. 31, 2009 discloses a steerable medical catheter for introducing a guidewire into a body of a patient and properly placing it. A steering mechanism is adapted to control longitudinal and rotational movement of the elongated member and to control off-axis deflection of its deflectable portion.

U.S. Publication No. 20120209302 published August 16, 2012 discloses a support catheter for stowing and exchanging guidewires and other working elements. A catheter body has a proximal region and a distal region with a single, contiguous lumen formed from the proximal region through the distal region.

U.S. Publication No. 2014033022 published Nov. 6, 2014 discloses multi-lumen catheter with separate distal tips extending from a distal end. The second tip section may include a first segment extending from the distal end of the catheter body to a transition segment and a second segment extending from the transition segment to a distal end of the second tip section.

U.S. Pub. No. 20140180089 published Jun. 26, 2014 relates to guidewires for intravascular procedures that include an electroactive polymer. The polymer reacts to an applied electrical potential by changing a dimension (e.g., contracting or expanding). Electroactive polymers can be disposed within the guidewires in helical, longitudinal, parallel, or coaxial patterns relative to the guidewire.

U.S. Pat. No. 4,960,411 issued Oct. 2, 1990 discloses a steerable catheter having distal and proximal ends and one or more lumens for introduction of a central guide wire, and a deflection wire axially extending the length of the catheter through a lumen with a closed end, with the distal end of the deflection wire embedded in a closed end.

U.S. Pat. No. 4,968,307 issued Nov. 6, 1990 discloses a vascular catheter for delivering a therapeutic fluid such as thrombolytic agents to a thrombus or clot in a patient's arterial system. The catheter has a thick-walled tubular body with a central lumen for advancement over a guidewire and a plurality of fluid delivering lumens disposed within the thick wall. Each of the smaller lumens has a single flow passageway to discharge therapeutic fluid to the exterior of the catheter.

U.S. Pat. No. 5,203,338 issued Apr. 20, 1993 discloses a vascular catheter having a distal region and proximal region, with the distal region reduced cross-sectional area smaller than that of the proximal region. The proximal region includes at least two lumens for accommodating a guidewire and a rotatable working element. The catheter body may be inserted over the movable guidewire, with the guidewire passing through a lumen in the distal region and the guidewire lumen in the proximal region. After positioning the catheter body, the guidewire can be retracted from the distal region and into the proximal region, leaving the lumen in the distal region available for the rotatable working element.

U.S. Pat. No. 5,219,335 issued Jun. 15, 1993 discloses an intravascular catheter comprising an elongate member having a proximal end and a distal end and first and second lumens. The first lumen communicates with a first proximal opening at the proximal end of the catheter. The second lumen terminates distally and communicates with the first lumen at a location proximal of the first distal opening.

U.S. Pat. No. 5,292,305 issued Mar. 8, 1994 shows a double-lumen angioscopy catheter which has separate passageways for a guidewire and an optic fiber bundle, which passageways are joined into a single tubular component for the distal portion of the catheter which enters the body. The guidewire can be used to maneuver the distal end of the optic fiber bundle so as to accomplish controlled inspection within the body vessel.

U.S. Pat. No. 5,458,584 issued Oct. 17, 1995 discloses an intravascular catheter having an enclosed proximal end and a reduced profile distal region. The catheter, which, may be connected to two or more proximal lumens, is loadable over a guidewire into a region of interest within a patient's vascular system. Means are provided to ensure that the guidewire enters into a desired one of the proximal lumens.

U.S. Pat. No. 5,531,700 issued Jul. 2, 1996 discloses a catheter with a proximal end having a first diameter and a distal region having a single lumen and a second diameter which is less than a first diameter. A distal guide wire exit port is disposed in the distal region of the catheter body within about 5 cm of the distal end. This allows the catheter to be used as a short lumen rapid exchange catheter. The catheter can also be provided with a proximal guide wire exit port in the proximal region so that the catheter can also be used as a long lumen rapid exchange catheter.

U.S. Pat. No. 5,620,417 issued Apr. 15, 1997 and U.S. Pat. No. 5,935,114 issued Aug. 10, 1999 disclose a rapid exchange delivery catheters for reducing exchange time when replacing one catheter with another in the vascular anatomy. The exchange catheter is used within a conventional guiding catheter where the tubular body is advanced through a distal end of the guiding catheter and into the coronary artery while the tapered fixture remains in the guiding catheter.

U.S. Pat. No. 6,117,128 issued Sep. 12, 2000 discloses a catheter comprising a flexible tube having a proximal portion with two lumens and a distal end portion with a flexible tube having a single lumen. The catheter has a tapered transition portion interconnecting the proximal and distal portions, in which the first and second lumens have openings communicating internally of the catheter to the single lumen in the distal end portion.

U.S. Pat. No. 6,120,516 issued Sep. 19, 2000 teaches a method for treating vascular occlusion, involving an intravascular catheter system for crossing a severe or total arterial occlusion; particularly, a steerable intravascular catheter providing a working element and the optical guidance thereof in crossing an occlusion.

U.S. Pat. No. 6,290,668 issued Sep. 18, 2001 shows a catheter having a proximal shaft with two lumens and a distal shaft with a single lumen connected to the proximal shaft. A guidewire and a light guide are received in the first and second lumens respectively, of the proximal shaft, either of which may be extended into the single lumen of the distal shaft, the distal shaft being sufficiently flexible to facilitate movement of the catheter through tortuous paths. A hub is provided having ports through which appropriate fluids may be applied to the lumens.

U.S. Pat. No. 6,394,141 issued May 28, 2002 discloses a single lumen to multiple lumen transition catheter. The single lumen is in fluid communication with each and every lumen comprising the multi-lumen portion.

U.S. Pat. No. 6,524,302 issued Feb. 25, 2003 discloses a multi-lumen catheter having a plurality of individual catheter tubes.

U.S. Pat. No. 6,730,037 issued May 4, 2004 shows a vascular catheter having low-profile distal end having a reduced cross-sectional area compared to that of the proximal region. The proximal region includes at least two lumens for accommodating a movable guidewire and a rotatable working element. The catheter body may be inserted over the movable guidewire, with the guidewire passing through a lumen in the distal region and the guidewire lumen in the proximal region. After positioning the catheter body, the guidewire can be retracted from the distal region and into the proximal region, leaving the lumen in the distal region available for the rotatable working element.

U.S. Pat. No. 7,018,358 issued Mar. 28, 2006 discloses a balloon catheter having an auxiliary lumen for guiding a functional measurement wire to a treatment site. The auxiliary lumen communicates with a working lumen that is configured to permit the advancement of a guidewire and the functional measurement wire, so that the guidewire and functional measurement wire may selectively be advanced to the treatment site during a surgical procedure.

U.S. Pat. No. 7,229,429 issued Jun. 12, 2007 discloses another multiple lumen catheter with an elongate body having proximal and distal ends that encloses two similar, longitudinally extending lumens separated by a septum, with a further lumen within the septum. A convergently tapered tip extending from the distal end of the body defines part of the further lumen that extends distally beyond the longitudinally extending lumens, while a connector at the proximal end of the body couples plural access tubes in fluid communication with respective individual of the lumens.

U.S. Pat. No. 7,875,019 issued Jan. 25, 2011 shows a connection system for multi-lumen catheter having a hub assembly and a connector assembly. The hub assembly has a cannula configured for insertion into a catheter lumen and the connector assembly. A collet is connected to the hub assembly and positioned about a distal portion of the cannula such that an inner surface of the collet is spaced from an outer surface of the cannula. The collar and collet include features that interact with one another to provide a locking engagement.

U.S. Pat. No. 7,901,395 issued Mar. 8, 2011 reveals a catheter having staggered lumens. A first lumen and a second lumen extend through the catheter tube along the longitudinal axis with each having an opening. The end of the catheter tube and the openings of the lumen may be disposed within the hub so that the opening of the first lumen is axially spaced with the end of the catheter tube.

U.S. Pat. No. 7,981,093 issued Jul. 19, 2011 discloses a method of making a multi-lumen catheter assembly to include a unitary portion and at least two distal end tubes extending distally from the unitary portion. The unitary portion includes an exterior having a generally circular or oval shape in cross section and includes at least two distal end tubes of generally semi-circular (or other) cross sectional shape extending longitudinally therethrough.

U.S. Pat. No. 8,162,891 issued Apr. 24, 2012 discloses another delivery and exchange catheter for storing guide. A single, contiguous lumen is formed from the proximal region through the distal region and has a larger proximal cross-sectional area than distal cross-sectional area.

U.S. Pat. No. 8,764,730 issued Jul. 1, 2014 discloses a vascular catheter having a flexible shaft, guidewire lumens passing through the shaft, and a positioning device for positioning the guidewires relative to an external lumen. The positioning device may be an expansible scaffold covered with a retractable sheath.

U.S. Pat. No. 8,801,691 issued Aug. 12, 2014 shows another delivery and exchange catheter for storing guidewire. A single, contiguous lumen is formed from the proximal region through the distal region and has a larger proximal cross-sectional area than distal cross-sectional area.

U.S. Pat. No. 9,125,683 issued Sep. 8, 2015 shows a catheter for insertion into vasculature of a patient to a target area in the vasculature, including a hollow inner shaft, a non-occluding self-expandable scaffold coupled to the distal end of the inner shaft and disposed at the distal end of the inner shaft, and a hollow outer shaft. The outer shaft is slidable over the inner shaft and scaffold such that the scaffold is in a non-expanded state when the outer shaft is around the scaffold.

U.S. Pat. No. 9,526,874 issued Dec. 27, 2016 discloses catheters for occluding, visualizing, irrigating, evacuating, and delivering agents to a treatment area. The catheter body comprises five lumens, first and second occlusion balloons coupled to the catheter body, an optional space-occupying balloon coupled to the catheter body and disposed between the first and second occlusion balloons, and an optional visualization means that enables visualization between the first and second occlusion balloons.

U.S. Pat. No. 9,579,485 issued Feb. 28, 2017 discloses another multi-lumen catheter with first and second lumens. The catheter body defines a distal tip region that includes a venous lateral opening that is in fluid communication with the first lumen and includes a distal-facing portion.

U.S. Pat. No. 9,358,037 issued Jun. 7, 2016 reveals methods and apparatus for centering a micro-catheter within a vasculature, allowing a guidewire to cross through a chronic total occlusion. The catheter may include a micro-catheter having a lumen, a distal opening and a distal end, and one or more guidewires for passing through the lumen.

U.S. Pat. No. 9,387,304 issued Jul. 12, 2016 discloses a multi-lumen catheter including a generally cylindrical body enclosing a first and second lumen separated by a generally planar septum. A first and second tip section extend from a distal end of the body. U.S. Pat. No. 9,358,037 issued Jun. 7, 2016 reveals methods and apparatus for centering a micro-catheter within a vasculature, allowing a guidewire to cross through a chronic total occlusion. The catheter may include a micro-catheter having a lumen, a distal opening and a distal end, and one or more guidewires for passing through the lumen.

Despite the several prior art attempts at multi-lumen catheters, no known prior art catheter allows a pair of guidewires first inserted through a proximal region of the catheter to be preloaded within the catheter concurrently while the different guidewires sequentially probe the target.

More importantly, by gentle retraction of one guidewire, the next preloaded guidewire can be quickly engaged to steer into the branch lumen or probe the blockage of the target lumen. The multi-lumen design offers simultaneous probing of the blockage with two or more guidewires therefore improving on procedural speed and accuracy.

### SUMMARY OF THE INVENTION

This invention provides a multi lumen intravascular support and vascular blockage crossing catheter. The catheter can control and direct more than one guidewire into critical areas requiring treatment for blockage. The present multi-lumen design facilitates sequential probing of blockages with successive guidewires which may be of different diameters and tip weights.

The multi-lumen catheter is constructed such that two or more guidewires can be simultaneously preloaded and sequentially extended through the catheter, allowing the operator to rapidly switch between the preloaded guidewires, each with different characteristics and steering or crossing profiles. This construction allows a first guidewire to probe and then be withdrawn from the blockage, followed by a second guidewire already substantially lodged within the catheter that is directed through substantially the same pathway into the blockage. In other words, the surgeon may withdraw one guidewire from the blockage, followed by rapid insertion of a second guidewire of a different diameter into the same portion of the blockage already probed by the first guidewire.

The current invention obviates the need for multiple and lengthy guidewire exchanges, since it facilitates simultaneous insertion and use of different guidewires with different characteristics such as stiffness and diameter. The multi-lumen catheter concept also increases tracking support, because two or more wires are preloaded within the catheter.

A novel junction arrangement at the distal end of the catheter makes it easy for the first probing guide wire to be quickly retracted, allowing the second preloaded guidewire to be advanced through the freed distal junction and to exit the catheter tip at an angle different from the angle formed by the first wire. When attempting to cross a difficult vascular blockage, the second guide wire tip may thus create a different channel into the blockage and facilitate crossing through the length of the blockage.

The multi-lumen catheter concept also increases tracking support, because two or more wires are preloaded within the catheter. With the first preloaded wire serving as support, the next guidewire will retain better torque characteristics to be quickly steered into a branch vessel that arises at a steep angle, such as the anterior or the posterior tibial artery. At present, one has to completely remove one guidewire and then insert another through a single lumen catheter. The multi-lumen design obviates the need for multiple and lengthy guidewire exchanges, as it offers rapid and sequential probing of the blockage with two or more preloaded guidewires, therefore improving on procedural speed, accuracy and success.

Furthermore, the preferred catheter allows for catheter tip deflection based upon the weight of the guidewire. This deflection and the ability to control flexion facilitates easier crossing, shorter procedure times, and less radiation exposure. Further, costs are reduced as fewer guidewires are necessary and other ancillary costs are reduced.

Thus, a fundamental object is to provide a catheter for treating difficult vascular blockages.

It is also an object to provide a catheter of the character described that is configured such that multiple diameter guidewires may be directed through various lumens to arrive precisely at an intended target blockage.

Another basic object of the present catheter is to facilitate quick and accurate probing of difficult vascular blockages with two or more guidewires of different characteristics.

It is also a goal to facilitate rapid exchange between preloaded guidewires with different characteristics and diameters.

Another object of this catheter is to facilitate simultaneous preloading of different guidewires with different characteristics such as stiffness and diameter.

Another object is to provide a multi-lumen catheter of the character described that enables the use of guide-wires of different diameters, stiffness and tip weight to aid in treating difficult vascular blockages.

Another object is to allow alternating maneuvers between different guidewires through the distal catheter region to more effectively treat vascular blockages.

It is also an object to reduce time-consuming guidewire exchanges during surgery.

These and other objects and advantages of the present invention, along with features of novelty appurtenant thereto, will appear or become apparent in the course of the following descriptive sections.

### BRIEF DESCRIPTION OF THE DRAWINGS

In the following drawings, which form a part of the specification and which are to be construed in conjunction therewith, and in which like reference numerals have been employed throughout wherever possible to indicate like parts in the various views:

FIG. 1 is a longitudinal side elevational view of a preferred multi-lumen catheter constructed in accordance with the best mode of the invention;

FIG. 2 is an enlarged sectional view of distal tip taken generally along line 2-2 of FIG. 1;

FIG. 3 is an enlarged sectional view of the catheter intermediate elongated tube portion taken generally along line 3-3 of FIG. 1;

FIG. 4 is a longitudinal sectional view of the preferred multi-lumen catheter, with portions thereof broken away for clarity;

FIG. 5 is a substantially enlarged portion of FIG. 4, showing the right half thereof;

FIG. 5A is an enlarged, partially exploded isometric view of the intermediate junction region, detailing the preferred transition fixture;

FIG. 6 is a substantially enlarged portion of FIG. 4, showing the left half thereof;

FIG. 7 is an enlarged, fragmentary sectional view of the catheter distal end derived from circled region “7” in FIG. 1;

FIGS. 8A-8C are enlarged, fragmentary longitudinal sectional views of alternative catheter guidewire junction portions;

FIG. 8D is an enlarged sectional view taken along line 8D-8D of FIG. 8C;

FIG. 8E is a fragmentary isometric view of an alternative catheter;

FIG. 9 is an enlarged, fragmentary sectional view detailing the catheter transition fixture and the bordering catheter regions;

FIG. 10 is a greatly enlarged, vertical sectional view of the transition fixture taken generally along line 10-10 of FIG. 9;

FIGS. 11-19 are combined sectional and diagrammatic views showing use of the catheter in a process involving sequential insertion of different guidewires; and,

FIGS. 20 and 21 are combined sectional and diagrammatic views of the preferred catheter with the angularity exaggerated for clarity, showing how a second guidewire, for example, may be directed outwardly at an acute angle relative to the first guidewire, with the distal catheter region assuming an angular orientation that enables enhanced, more vigorous unblocking.

### DETAILED DESCRIPTION OF THE DRAWINGS

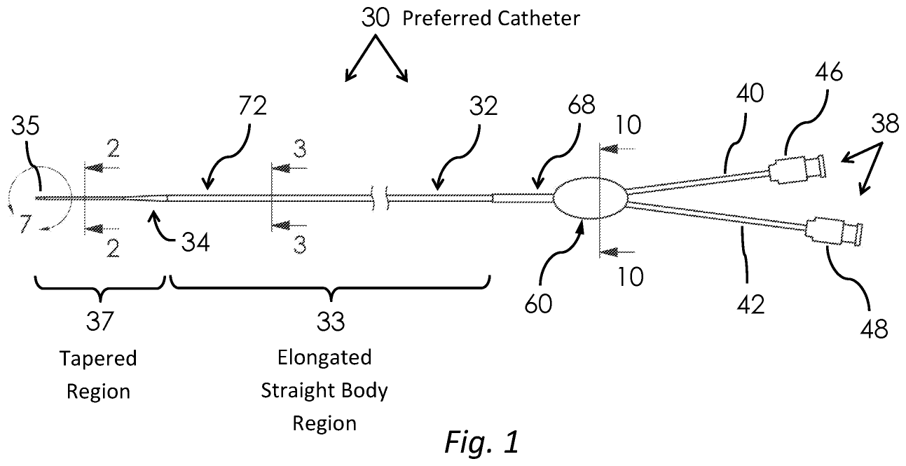

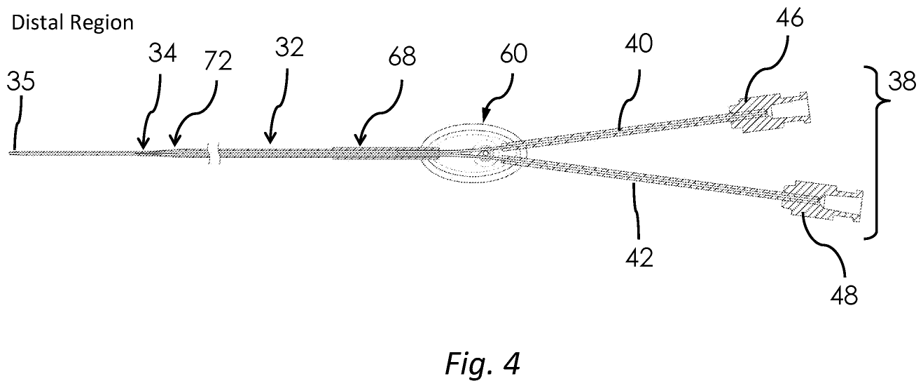

With initial reference now directed to FIGS. 1-7 of the appended drawings, the present multi-lumen catheter 30 is to be used in an over-the-wire type mode. It allows the concurrent use of two guidewires of different diameters and weights, as explained below, and it is both a support and crossing catheter.

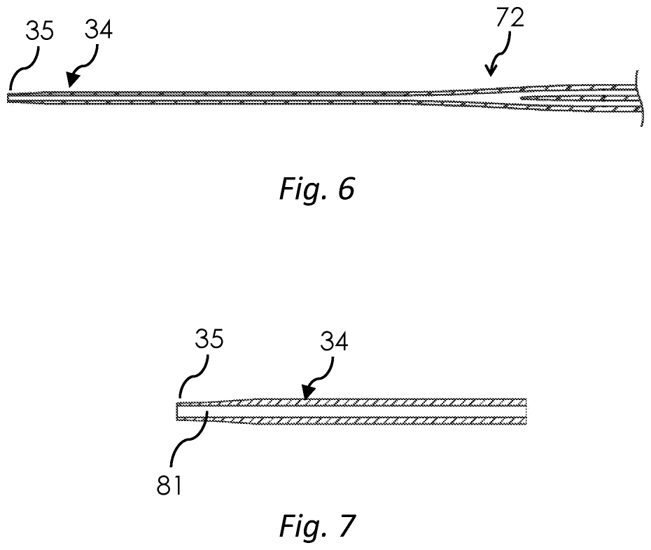

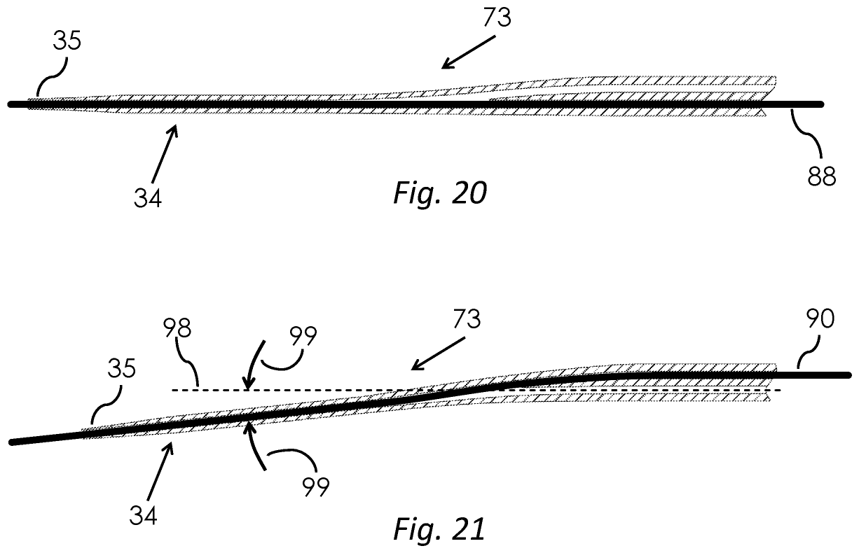

The preferred catheter 30 comprises an elongated, catheter body of medical grade elastomer or thermoplastic known in the art. Preferably its major tubular portions are made of extruded thermoplastic polymer tubing. The body is formed by a tubular distal region 34 (FIGS. 1, 6) which is part of the tapered catheter region 37 (FIG. 1). Region 34 is specially designed for guidewire control, and for directing the guidewires as explained hereinafter (i.e., FIGS. 20, 21). Tubular distal region 34 terminates in a reduced diameter, penetrating tip 35 (FIG. 6) whose diameter will accommodate the diameters of the different guidewires. The tapered region 37 which ultimately communicates with a spaced-apart proximal region generally designated by the reference numeral 38. It is important that the diameter of the tip 35 be as close as possible to the diameter of wires traversing through it, as explained below.

There is an elongated straight body region 33 between the tip 35 and the transition fixture 60. The proximal end 38 of the catheter is fitted with two or more ports, designed to accommodate each the insertion of a specific guidewire. The proximal region 38 accommodates guidewires of different characteristics such as caliber, weight, stiffness steerability, torquability and trackability. Preferably the catheter proximal region 38 (i.e., FIG. 1) comprises a pair of converging lumen tubes 40, 42 comprising part of the plastic catheter body. The inputs of tubes 40 and 42 are respectively connected to suitable fittings, such as Luer fittings 46 and 48, that enable the connection of a syringe for pre-treatment flushing. These can connect to Tuohy-Borst adapters that provide guidewire input ports.

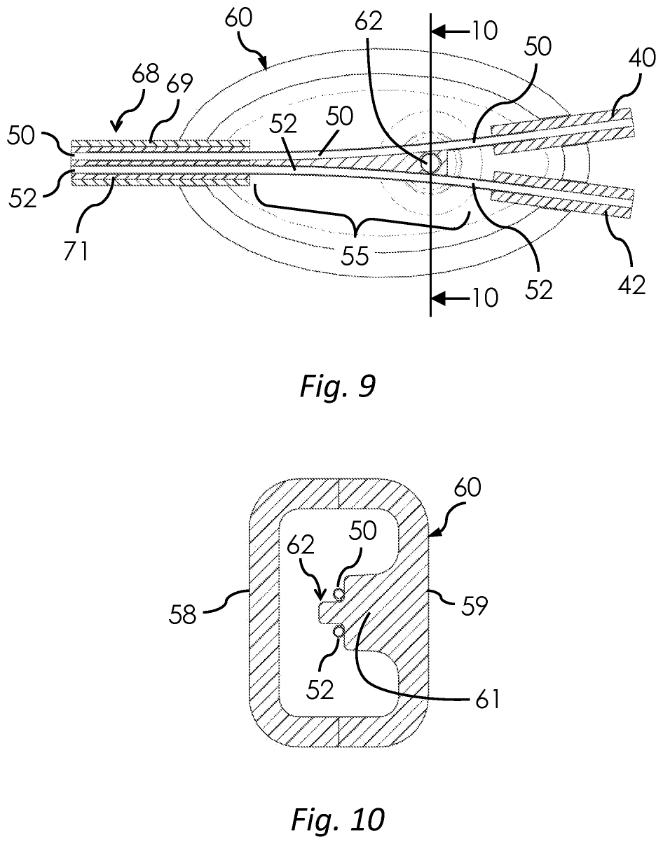

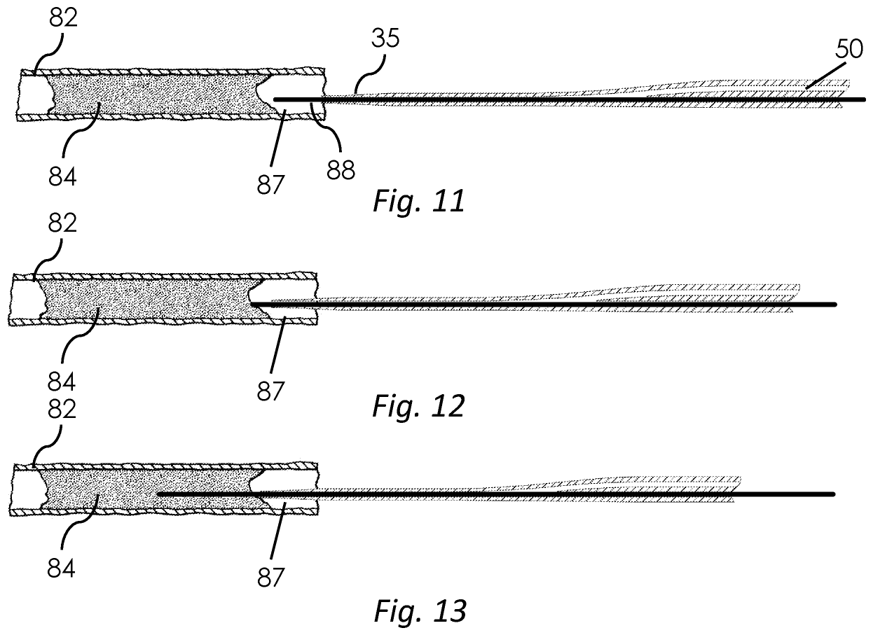

The internal, concentric guidewire-receiving connecting lumens 50, 52 respectively extend coaxially through catheter body tubes 40 and 42, and enter and travel through an intermediate transition fixture 60 (i.e, FIG. 9) proximate the transition guidewire region 55 (FIG. 9). Lumens 50 and 52 can slidably receive and pass guidewires through the fixture and out of the catheter distal tip 35. The diameters of connecting passageways 50 and 52 may be different. Guidewire progress through the latter regions out of the tip 35 and into a target vascular blockage are diagrammed in FIGS. 11-19 as discussed hereinafter. The lumens 40 and 42 are directed into and through a unique transition fixture 60 (FIGS. 9, 10) that controls their spacing and geometry, in effect allowing lumens 50 and 52 to enter fixture 60 at an acute angle (FIG. 9) and exit substantially parallel with one another. The inner lumens 50, 52 contact and are separated by a molded divider 62 (FIG. 10), which conforms the lumens 50, 52 into a generally parallel relationship within transition region 55 prior to exiting fixture 60.

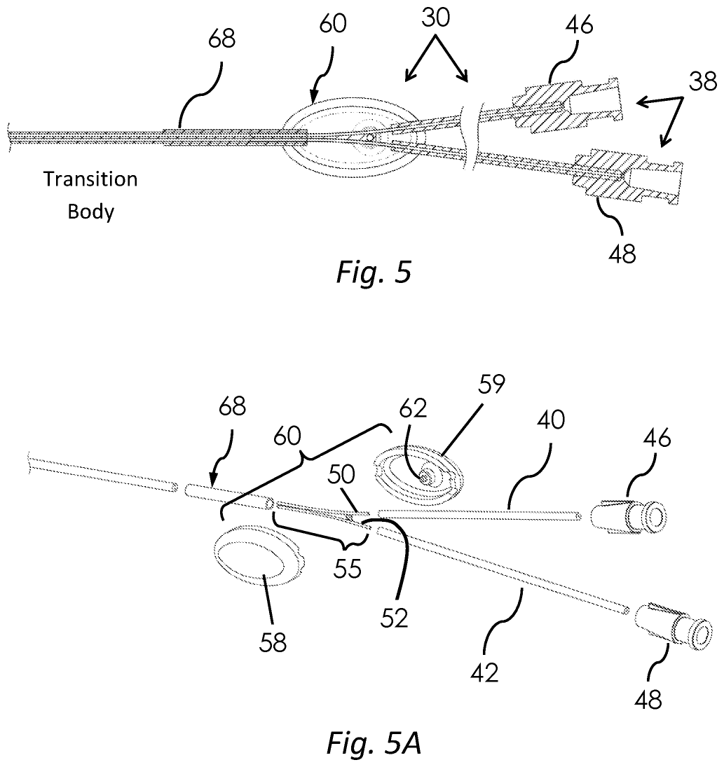

The inner lumens 50, 52 are designed to accommodate a variety of specialty guidewires that may vary in size and function. Lumens 50 and 52 and gently angled relative to each other within the transition fixture 60 in region 55 (FIG. 9). Fixture 60 non-destructively and gently conducts the somewhat stiff guidewires to their exiting destination. Strain relief section 68 (FIG. 9) preferably comprises a strain relief section made of an outer tubing portion 69, also made of thermoplastic polymer tubing, which concentrically surrounds inner tubing 71. Section 68 comprises outer concentric portion 69 that coaxially surrounds tube 71 (FIG. 5) strengthens the tubing overall, and provides strain relief. A medial catheter body portion 32 (FIGS. 1 and 3) traversing catheter straight section 33 (FIG. 1) is of generally round vertical profile, supporting twin, parallel passageways 50A, and 52A (FIG. 3) for lumens 50, 52. Upon exiting the transition fixture 60 the lumen separation depicted in FIG. 3 preserves substantially parallel alignment.

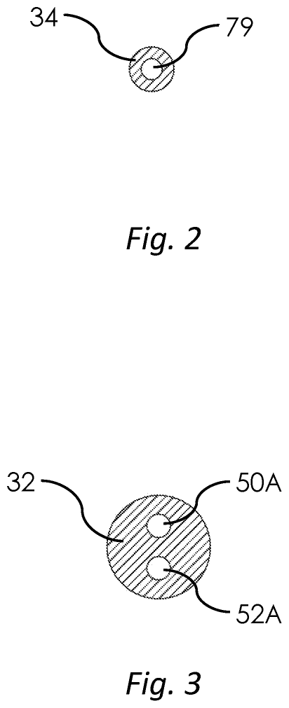

The inner transition lumens 50, 52 exit the transition fixture 60 within and through tubular strain relief section 68 (FIGS. 1, 5, 9) and extend through catheter body section 32 (FIGS. 1 and 3) within passageways 50A and 52A. They then converge and traverse single lumen passageway 79 (FIG. 2) in catheter tubular distal region 34 (FIGS. 1, 2) which leads to the distal tip 35 of the catheter.

In FIG. 2 it is seen that the reduced diameter distal end 34 proximate the tapered tip 35 contains the inner concentric final passageway 79, into which either of the guidewires may be directed.

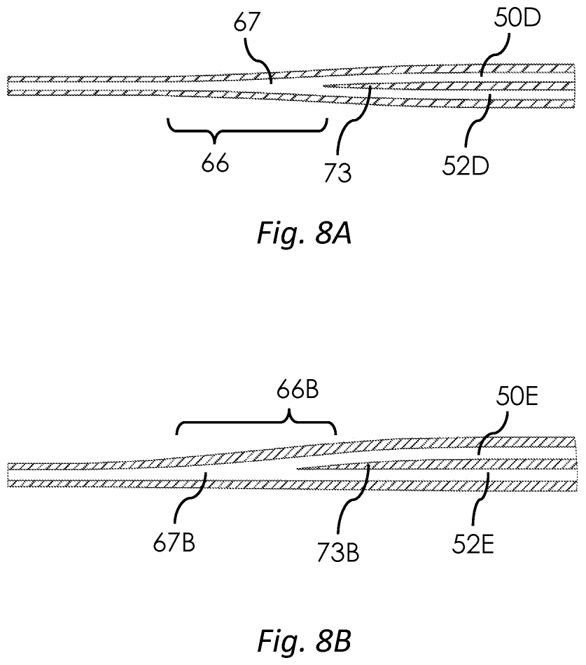

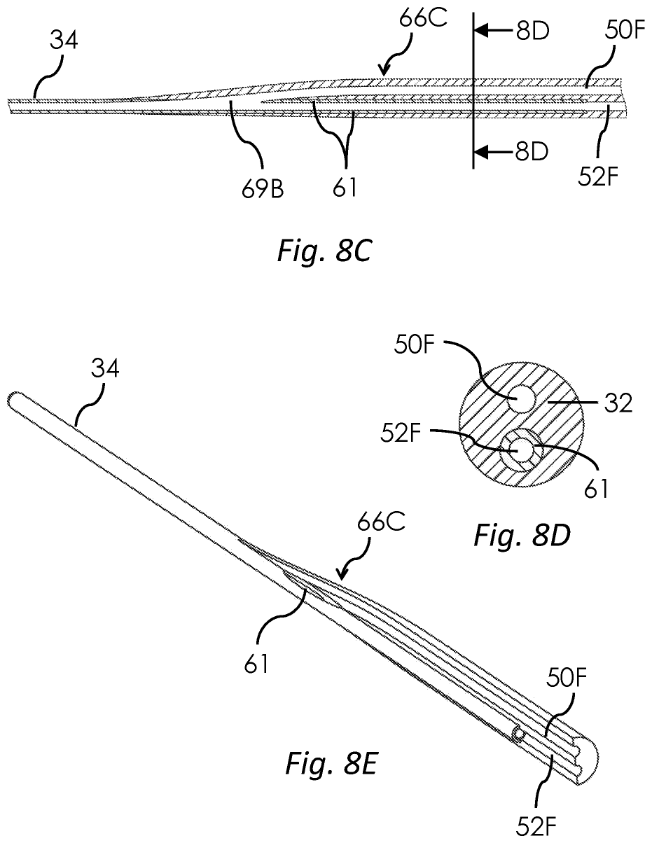

The lumen convergence region may have alternative designs. FIG. 8A details one embodiment of a lumen convergence region 66. In this embodiment the lumens have been designated 50D and 52D, and they are separated by a wall 73. A common distal passageway 67 extends through to the end portions of the catheter.

FIG. 8B shows a preferred lumen convergence region 66B where a different plastic is used. Here the lumens, designated 50E and 52E for clarity, are separated by wall 73B and converge in passageway 67B. The cross section is similar to that seen in FIG. 3. The lumen 52E is substantially colinear with passageway 67B. However lumen 50 enters region 66B at an angle, which helps direct and control the larger and stiffer second guidewire during use. This angularity is important for the proper functioning of the best mode of the invention.

FIG. 8C shows an alternative convergence region 66C. A guidewire traversing through lumen 52F runs substantially horizontally towards region 69B. As described later, an upper guidewire traversing lumen 50F travels through convergence region 66C but angles downwardly (i.e., as viewed in FIG. 8C) and straightens out through region 69B. Optionally there is an encircling reinforcement 61 (FIG. 8D) adding more resilience to the converging region 66C.

Referencing FIGS. 5, 5A and 9-10, the transition fixture 60, preferably injection molded from plastic, comprises a pair of body segments 58, 59 that are preferably cemented together. Body segment 59 comprises an inner salient 61 projecting towards the fixture interior, from which the divider 62 integrally projects. Lumens 50, 52 are separated by contact with the divider 62, within fixture 60, to preserve their gentle transitioning from a generally angular relationship entering the fixture 60, to the generally parallel relationship assumed when exiting the fixture 60.

Referencing FIGS. 11-19, a portion of an obstructed human vessel 82 is seen to have an inner difficult vascular obstruction or blockage, that is to be engaged and removed or dislodged by probing guidewires during surgery. In a first stage of operation, a first guidewire 88 (FIG. 11) may traverse the distal catheter region substantially horizontally and parallel with the axis of the catheter. A guidewire 88 (FIG. 11) may traverse the catheter substantially horizontally, traveling through the convergence region 66C (FIG. 8) and through the single passageway region, out of the tip, and into blockage 84 (i.e., FIG. 11-13). The guidewire tip exits the catheter tip 35, traverses the interior 87 of the vessel 82, contacts the vascular blockage as in FIGS. 12 and 13, and penetrates and engages the blockage as in FIG. 13.

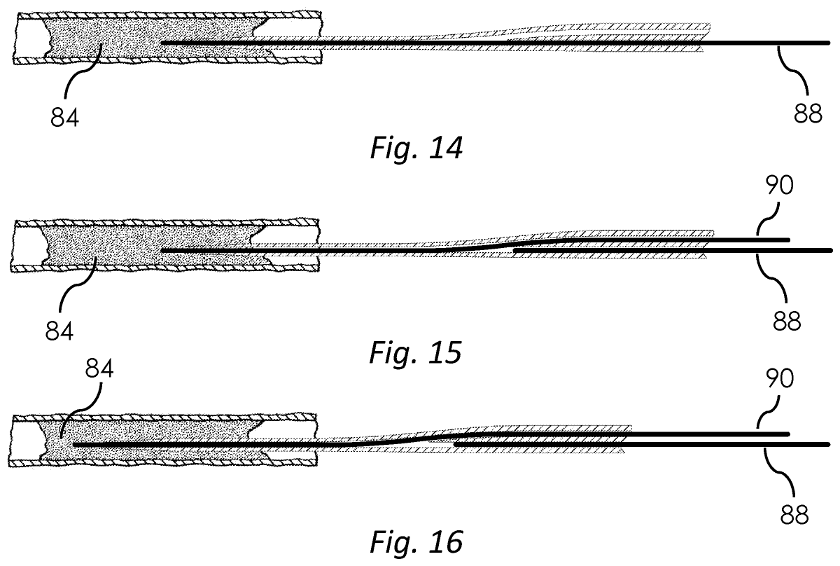

As the first guidewire 88 progressively engages the blockage 84 (i.e., FIGS. 11-13), the tapered catheter tip 35 will follow into the blockages, as in FIG. 14. It is important that the distal catheter tip 35 be tapered as much as possible to be very close in diameter to the guidewire diameter; this enables portions of the catheter tip 35 to smoothly enter the vascular blockage as the guidewire tip forcibly traverses that region. Furthermore, after tip 35 enters the blockage 84 (FIG. 11), it will remain in the blockage after the withdrawal of guidewire 88. With the catheter tip 35 plunged into the blockage as in FIG. 14, control of the second guidewire 90 is enhanced. Thus, when second guidewire 90 is thereafter inserted (FIGS. 15, 16) it will exit tip 35 in the exact blockage region previously engaged by the now-withdrawn first guidewire 88. Thus targeting and “aiming” in conjunction with the second guidewire is eased and simplified through the depicted catheter construction.

Importantly the first guidewire 88 (FIG. 14) may be retracted somewhat and withdrawn, to the position illustrated in FIG. 15. With guidewire 88 withdrawn to a non-interference position (FIG. 15), the second guidewire 90 may be deployed as in FIGS. 15 and 16 without completely removing guidewire 88 from the catheter. Further, since the tip 35 had been embedded within the vascular blockage as it followed guidewire 88, guidewire 90 will emerge from catheter tip 35 in the exact region originally targeted in the vascular blockage during initial penetration by the first guidewire 88.

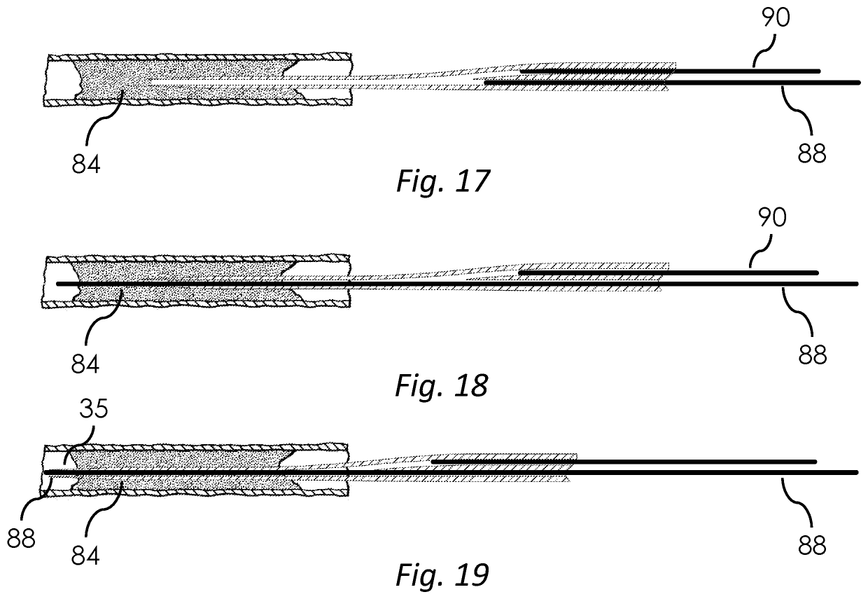

As mentioned, partial withdrawal of the first guidewire 88 provides clearance for the second guidewire 90. Once the second guidewire has been partially extracted, as in FIG. 17, the first guidewire 88 may once again penetrate the CTO, exiting the tip 35 well within the obstructed region (FIG. 18). Guidewire 88 and catheter tip 35 may eventually clear the blockage as in FIG. 19. Penetration of the vascular blockage may also reoccur by guidewire 90, which may be of a larger diameter than guidewire 88 and it may be stiffer or more resilient as well. FIGS. 18 and 19 detail how alternating use and switching between the guidewires is possible. FIG. 19 reveals that the catheter tip and the first guidewire 88 may exit the blockage 84 completely at the blockage distal end.

Angular orientation of the two guidewires 88 and 90 and guidewire control during operation is significant in tubular distal region 34 (FIGS. 20, 21) that is spaced from the previously described convergence region 66C (FIG. 8C). It is noted in FIG. 20 that the first guidewire 88 discussed above normally traverses the length of the catheter substantially horizontally, forming an axis 98 (FIG. 21) indicated by dashed lines. Because of the angularity of the second guidewire 90 as depicted in FIG. 21, which is caused by the construction of region 34 and the convergence region, designated here by the reference numeral 73, guidewire 90 exits the catheter 21 at an acute angle relative to the first guidewire path, as indicated by reference arrows 99 (FIG. 21). In FIG. 21 this angularity is exaggerated for clarity. At this time the distal catheter region 34 will also assume an angular orientation that enables enhanced, more vigorous engagement within the vascular blockage.

For example, with a second guidewire engaged as in FIGS. 15, 167, subsequent twisting or relative rotation of the guidewire 90 by the surgeon, will more vigorously assault the vascular blockage. The tubular distal region 34 thus allows torque transmission to the catheter tip via a stiffer and inclined guide wire 90.

Operation:

The first guidewire 88 is inserted into the catheter via a proximal end port through a fitting 46 or 48. It enters the transition fixture 60, passes through the terminal 68, exits the catheter through the angularity distal portion 34, and exits distal tip 35 (FIG. 7). The common or “output” lumen 81 (FIG. 7) is approximately several centimeters in length and is fitted to the distal end of the catheter exactly tapered to the size of exiting guidewire. The next guidewire is loaded into its inner lumen via the next proximal entry port (i.e., Luer fitting 48) and advanced slightly proximal to the converged inner lumen towards the distal tip of the catheter. By simply retracting the first guidewire into its inner lumen, the next guidewire is advanced through the inner lumen to eventually exit the catheter. It is to be noted from FIG. 20 that the first guidewire 88 may assume a rather flat or parallel travel through the catheter. However, the stronger and perhaps slightly bent guidewire 90, separated from the passageway occupied by guidewire 88, can arc or curve the catheter distal end as it exits the catheter.

With this concept, several guidewires with different characteristics such as size, steerability, crossing function or other parameters are easily advanced one at a time into the vessel lumen by simply retracting the previous guidewire, obviating the need for time-consuming guidewire exchanges. Physician interventionists, such as interventional cardiologists, interventional radiologists or vascular surgeons would use the catheter when attempting to treat a blockage in a vessel or a hollow viscus under fluoroscopic guidance.

From the foregoing, it will be seen that this invention is one well adapted to obtain all the ends and objects herein set forth, together with other advantages which are inherent to the structure.

It will be understood that certain features and subcombinations are of utility and may be employed without reference to other features and subcombinations.

As many possible embodiments may be made of the invention without departing from the scope thereof, it is to be understood that all matter herein set forth or shown in the accompanying drawings is to be interpreted as illustrative and not in a limiting sense.

What is claimed is:

1. A multiple lumen catheter adapted to treat difficult vascular blockages, the catheter comprising:
an elongated, catheter body of medical grade thermoplastic tubing, the body comprising a proximal input region and a spaced-apart, distal region with a blockage penetrating tip;
the proximal end of the catheter comprising a pair of angled, tubular input lumens that are respectively connected to suitable fittings that provide guidewire input ports;
a guidewire transition region adapted to slidably receive and pass guidewires and to gently transform them from an incoming angular relation to a generally parallel relationship;
a strain relief section exiting the guidewire transition region;
the strain relief section coupled to a single elongated body lumen with a guidewire divider therewithin;
a reduced diameter distal segment near the distal region of the catheter; and,
the reduced diameter distal region terminating in a tapered tip from which guidewires exit.

2. The catheter as defined in claim 1 wherein the input lumens traverse a transition fixture proximate the transition guidewire region and are realigned into a generally parallel relation.

3. The catheter as defined in claim 2 wherein the transition fixture comprises an internal divider for transforming guidewires into a generally parallel orientation relative to one another.

4. The catheter as defined in claim 3 wherein the transition fixture is injection molded and comprises a pair of halves adapted to be coupled together, at least one of the halves comprising a projecting divider for gently transitioning guidewires from a generally angular relationship entering the transition fixture to a generally parallel relationship when exiting the fixture.

5. A multiple lumen catheter allowing sequential probing of difficult vascular blockages with successive guidewires which may be of different characteristics, the catheter comprising:
an elongated, catheter body of medical grade thermoplastic tubing, the body comprising a proximal input region and a spaced-apart, distal region;
at least a pair of angled lumens at the proximal end;
the proximal end of the catheter comprising suitable fittings for providing guidewire input ports;
a transition guidewire region adapted to gently transform incoming angled lumens to a generally parallel relationship;
a distal region terminating in a tapered vascular blockage penetrating tip from which guidewires may exit;
a first lumen entering said angle control distal region substantially horizontally;
a second lumen entering said angle control region at an acute angle relative to said first lumen;
the first lumen initially conducting a first guidewire into said a target vascular blockage;
wherein after retraction of said first guidewire said catheter tip may remain within the blockage;
the second angled lumen conducting a second guidewire into said vascular blockage after said first guidewire has been at least partially retracted, the second lumen conducting said second guidewire at an angle relative to said first guidewire; and,
whereby said second guidewire penetrates said blockage at said target region.

6. The catheter as defined in claim 5 wherein the catheter comprises a convergence region, wherein a first guidewire traverses the catheter substantially horizontally and wherein a second guidewire forms an acute angle relative to the first guidewire.

7. The catheter as defined in claim 5 wherein the input lumens traverse a transition fixture proximate the transition guidewire region and are realigned into a generally parallel relation.

8. The catheter as defined in claim 7 wherein the transition fixture comprises an internal divider for transforming guidewires into a generally parallel orientation with one another.

9. The catheter as defined in claim 8 wherein the transition fixture is injection molded and comprises a pair of halves adapted to be coupled together, at least one of the halves comprising a projecting divider for gently transitioning guidewires from a generally angular relationship entering the fixture to a generally parallel relationship when exiting the fixture.

10. The catheter as defined in claim 9 wherein the catheter comprises a convergence region, wherein a first guidewire traverses the catheter substantially horizontally and wherein a second guidewire forms an acute angle relative to the first guidewire.

11. A multi-lumen vascular support catheter for preloading at least a pair of guidewires, the catheter comprising:
an elongated flexible body of predetermined length;
the body comprising a proximal region and a distal region;
wherein the proximal region comprises at least two inner guidewire lumens that merge into a common lumen in the distal region;
wherein a first guidewire lumen is colinear with said common lumen;
wherein a second guidewire lumen merges with said first guidewire lumen in a distal region of said catheter and forms an acute angle relative to said first guidewire lumen.

12. The catheter as defined in claim 11 wherein the diameter of said first and second guidewire lumens is different.

13. The catheter as defined in claim 12 wherein the distal catheter tip is substantially equal to the diameter of at least one guidewire.

14. The catheter as defined in claim 12 wherein the proximal region allows the passage of guidewires of different characteristics such as caliber, weight, stiffness steerability, torquability and trackability.

15. The catheter as defined in claim 12 wherein a guidewire tracking through said angled second lumen will deflect the distal catheter tip at an angle relative to the other guidewire.

16. The catheter as defined in claim 11 further comprising a transition fixture for transforming incoming guidewires into a generally parallel orientation with one another.

17. The catheter as defined in claim 16 further wherein said transition fixture is injection molded and comprises a pair of halves adapted to be coupled together, at least one of the halves comprising a projecting divider for gently transitioning guidewires from a generally angular relationship entering the fixture to a generally parallel relationship when exiting the fixture.
